SEQUENCE LISTING (29 sequences)

SEQ ID NO: 1 - DNA, 1041 bp, synthetic construct
  source 1..1041: mol_type = other DNA
atgacagacaagagccaacaattcgccagcgacaactattccggtatctgccccgaagcctgggtggcgatggagaaagccaaccgcggccacgaccgcgcctacggcgacgaccagtggaccgagcgtgcatcggagtacttccgcaatctgttcgaaaccgactgcgaggtgttctttgccttcaacggcaccgcggccaactccctggccctggcatccctgtgccagagctatcacagcgtgatctgctccgagaccgcccacgtcgaaaccgacgaatgcggtgcaccggagtttttctccaacggctccaagctgctgacggcggccagcgtcaacggcaagctgacgccacagtcgatccgcgaagtggcgctcaaacgccaggatatccactaccccaagccgcgcgtggtgaccattacccaggccaccgaagtgggcacggtgtaccgccccgacgagctgaaggcgatcagcgccacctgcaaggagctgggcctgaacctgcacatggacggcgcacgctttaccaatgcctgtgcgttcctgggctgcagcccggccgaactgacctggaaggccggtgtggatgtgctgtgctttggcggcaccaagaacggcatggcggtgggcgaagcgattctgttcttcaaccgccagctggccgaagacttcgattatcgctgcaagcaggccgggcaactggcgtcgaagatgcgcttcttgtcggcgccctgggtcgggctgctggaagatggcgcatggttacgccatggcaaccacgccaatcattgcgcgcaactgctggcatcgctggtcagtgacctgccgggggtagagctgatgttcccggtggaggccaacggggtgttcctgcagatgccagagcacgccatcgaggcgctgcggggcaagggctggcgcttctatacctttatcggcagcggtggcgcgcgcttcatgtgctcgtgggataccgaagaagcgcgggtgcgtgagctggcggcggatatccgcacgatcattggtggctga

SEQ ID NO: 2 - DNA, 1002 bp, synthetic construct
  source 1..1002: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacacccacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgttaa

SEQ ID NO: 3 - DNA, 1545 bp, synthetic construct
  source 1..1545: mol_type = other DNA
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 4 - DNA, 1032 bp, synthetic construct
  source 1..1032: mol_type = other DNA
atgtcgacctcaccccaccgccccgctcatctgacgatcaacggcgagccgattccgacgctcgacgaaatcgccgcgcagcacttcgcgctcacgccgtgggtcacgcgtacgccggtgttcgagcgcgccgatctgccatcgctcgacaacacgcacgtgaacttcaagttcgagctgctgcaggcgagcggcagcttcaaggcgcgcggcgcgttcacgaacctgctcgcgctcggcgaggacagaaagcgcgcgggcgtcacctgcgtgtcggcgggcaatcatgcggcggccgtcgcttatgcggcgatgcggctcggcacgagcgcgaaggtcgtgatgttcgacaccgcgagcccggcgcgcgtcgagctctgccaccagtaccgcgcggaggtcgtgatggcgtcgagccccgccgacgcgttcgagatcgtccggcgaatcgaggccgaggaaggccgcttcttcgtgcatccgttcaacggctatcacacgattctcggcaccgcgacgctcggctacgaatgggcgacgcagacgcccgacctcgatgcggtgatcgtgccgatcggcggcggcggcctcgccgcgggcacctcgaccgcgctgcggcttgcgaatccgcgcgtgcacgtgtacggcgtcgagccggaaggtgcggatgtgatgagccgcagcttcgccgcgaatcgcccggtccgcatgggcgcgatgcatacgatcgccgattcgctgatggcgccgcacaccgaggagtacagctacgagctgtgccgccgccatgtcgaccggatcgtcaccgtctcggacgacgcgctgcgagcggcgatgctctatttgttcacgcatctgaaagtggcggtcgagcccgcgtgcgcggccgcgacggccgccctcctcgggccgttgcgcgagtcgctgcaaggcaagcgtgttggcgtgctgctgtcgggcaccaacatcgattcgacggcgctcgccacgcatctcgagcatgctcggctgcatcgcatgtcgtggccgtga

SEQ ID NO: 5 - DNA, 387 bp, synthetic construct
  source 1..387: mol_type = other DNA
atgagcaaaactatcgcgacggaaaatgcaccggcagctatcggtccttacgtacagggcgttgatctgggcaatatgatcatcacctccggtcagatcccggtaaatccgaaaacgggcgaagtaccggcagacgtcgctgcacaggcacgtcagtcgctggataacgtaaaagcgatcgtcgaagccgctggcctgaaagtgggcgacatcgttaaaactaccgtgtttgtaaaagatctgaacgacttcgcaaccgtaaacgccacttacgaagccttcttcaccgaacacaacgccaccttcccggcacgttcttgcgttgaagttgcccgtctgccgaaagacgtgaagattgagatcgaagcgatcgctgttcgtcgctaa

SEQ ID NO: 6 - DNA, 1479 bp, synthetic construct
  source 1..1479: mol_type = other DNA
atggcggtatttgccagtgactcttttggccagctgaaagtggagaaaattatgactgcccagtggaaccactacattaacggggaatacgtatcacccgaatctgaagagtatatccacgagttcatcccaaccacggctttgccgggtgactcaatcgcaaggggctcggcagctgacgttgataaggctgttcgtgccgcggcagcggctcagcctgcctggaatgcacgcaagccaattgagcggggtcgtatccttctcgccatagctcgtttggttcgcgccaacgcagcggctttctgcgccaaagaagcggaagaaactggcaagcctctgaagatggccgcctttgagatcgaggcatgtgctcagtactttgagtattacggcggtttggcgacagccatccagggcgaaaccatcaacctcggccccagctaccacgcctataccacccgagagccatttggagtggtgggggtcatcctgccgtggaattcgccactgaaccaagctgggcgagccattgccccggcattggttagcgggaacaccgtggtggtcaaaccttcagagttcacctcggtgacgatgctccagttcgcggaactggttgtgaaagaggcagggttgccaccaggcgtattgaacgtggttaccggcaccggtaaggaaaccggtgagcctctggttaaacaccctctgatccgaaaggttgctttcaccggttctgtccgtgccggacgggagatcggcaagctcgccgcagatcgcatcattccgctgtcgctcgaattgggcggcaaatccccgaacattgtcttcgaagacgcagatctggatcgagctgtcgcgggtagcgtctttgccttcaccgtcaacactggtcaagtctgtctcgccgggacccgttgcctggtgcatgagtcgatttttgaaaaattctccaagaagcttgccggtgctgtagaggcgcttcagttcagcgacggcgaaagcttcggtctcggccccctaacgaccaaggctcagtttgagcaggttcatcgttacaacgagctggccatccaggagggggctcattgcttggtcggtggggaagctccaagtgacaaaaccggctggtacgtacgacccaccgtctacaccaacgtcaacaactcgatgcggattgctcgggaagaaattttcggacccgttctggtactgattccgttcaaggacgaaaacgaggcggtggccatcgcgaatgactcggactacgggctcgcggctggcgtatggaccaccgatctggctcgcgcgcaccgcgtatccgctcaaatcgaagcgggccaggtgtacgtcaacgaatatccatcaggtggcgttgagactccattcggcggtttcaagcaaagcggccatgggcgcgagaagggcattgaagcactccaccattacacccaaacaaagacgaccatcatccgcatttaa

SEQ ID NO: 7 - DNA, 1287 bp, synthetic construct
  source 1..1287: mol_type = other DNA
atgcgcgcactggttagcaaagcacgcctggcacatagcgtgggtggtcgtgcgagccaggcgacccgttgcgcagcaaccattagcgcaagccgtgcaccggcccatctgggtgatgccctgcatgatgttgataccccggcactgattctggatctggatgcatttgatcgtaactgtgaaaaactgaaaggtgttatggcaggttttcctggtgttgcagttcgtccgcatgcaaaagcacataaatgtgcagaagttgcccgccgtcagctgcagctgctgggtgctaaaggtgtttgctgtcagaaagttattgaagcggaagcaatggcagaaggtggtgtttccgatctgctgctgagcaatgaagtgattgcacctcgtaaaatcgatcgtctggttggtctggcagcggccggtgcacgcgttggtgtgtgttatgaacgtgaagataatctgcgtcagctgaatgccgcagcagcagcacgtggtacacatctggatgttctggttgaactgaatgttggtcaggatcgttgtggtgttaatagcgccgatgaagttgttcagctggcccgtgcagccgccggtctggataatgttcgttttgcaggtattcaggcatatcatggtggtctgcagcatgttcgtgatccgcgtgatcgcgcacagcgtgttggtcaggttgttggtcgtgcacgtgcagcagttgatgcgctgaaagcagcaggtctgccatgtgataccgttacaggtggtggtacaggtacatatcgtgtggaagcagcaagtggtgtttttacggaagttcagccgggtagttttgcattttctgatgccgattatgcgcgtaatctgcaggaagatggtggtgtgggtgaatgggaacagagcctgtgggttctgacccaggtgatgagcgtgaccccggcacgtggtctggcagttgttgatgcaggtacaaaagcagtgtctctggatagtggtccgccgcgtctgccgccggcatttgaagctgcatacggtacaatgatggaatatggctctggtggtgatgaacatggtaaactgatgtggccgcagggtgcgtatcagctgccgatgtcactgcctgaagttggtagcctgctgctgctgcagccgggtcattgtgatccgacagttaatctgtatgattggctggtggccgcgcgtcgtcagcagggtggtcagcagcagggtggcgttgatggttggcgtgttgaagcagtttggccgattcgtggtcgtggtcctggccagtga

SEQ ID NO: 8 - DNA, 1353 bp, synthetic construct
  source 1..1353: mol_type = other DNA
atgggcaaactggacaaagcgagcaaactgatcgacgaagaaaataaatactacgcacgtagcgcgcgtatcaactactacaacctggtgattgaccacgcccacggcgcaaccctggtggatgtggatggaaataaatatatcgatctgctggcaagcgcaagcgcaattaatgttggtcatacccatgaaaaagttgtgaaagcaattgcagatcaagcacaaaaactgattcattataccccggcatacttccaccacgttccgggtatggaactgagcgaaaaactggcaaaaattgcaccgggtaatagccctaaaatggtgagttttggaaatagcggaagcgatgcaaatgatgcaattatcaaatttgcacgtgcatatacgggacgtcagtatattgttagttacatgggtagctatcacggaagcacctacggtagccagacactgtcaggttcaagcctgaatatgacccgcaaaatcggtccgatgctgccgagcgttgttcatgttccgtatccggattcatatcgcacctacccgggagaaactgagcacgatgttagcctgcgttattttaatgagtttaaaaagccgttcgagagctttctgcctgcagatgagaccgcatgtgttctgattgaaccgattcagggcgatggaggtattattaaagcaccggaagaatatatgcagctggtgtataaattttgtcatgaacatggtatcctgtttgcaattgatgaagttaatcaaggcctgggtcgtacaggaaaaatgtgggcaatccagcagtttaaagatattgaaccggatctgatgagcgttggtaaaagcctggcgtctggtatgccgctgagcgcagttatcggtaaaaaggaagttatgcagagcctggacgcacctgcacacctgtttaccaccgcaggtaatccggtttgtagtgcagcaagcctggcaactctggacgttattgaatatgaggggctggtggagaaaagcgcaaccgacggtgcatatgccaaacaacgttttctggaaatgcaacagcgccatccgatgatcggagatgttcgtatgtgggggctgaatgggggtattgaactggttaaagacccgaaaaccaaagaacctgatagtgacgcagcaaccaaagtgatttactacgcctttgcacacggggttgtaattattacgctggcaggcaacattctgcgctttcaaccgccgctggttatcccgcgtgaacaactggatcaggcactgcaggtgctggatgacgcctttaccgcagtcgaaaatggggaagttaccattccgaaagacaccggtaaaattggctggtaa

SEQ ID NO: 9 - DNA, 1143 bp, synthetic construct
  source 1..1143: mol_type = other DNA
atgagcctggtggaaaaaaccagcattattaaagattttaccctgtttgaaaaaatgagcgaacatgaacaggttgttttttgtaatgatccggcaaccggtctgcgtgcaattattgcaattcatgataccaccctgggtccggcactgggtggttgtcgtatgcagccgtataatagcgttgaagaagcactggaagatgcactgcgtctgagcaaaggtatgacctataaatgtgcagcaagcgatgttgattttggtggtggtaaagcagttattattggtgatccgcagaaagataaaagcccggaactgtttcgtgcatttggtcagtttgttgatagcctgggtggtcgtttttataccggtaccgatatgggtaccaatatggaagattttattcatgcaatgaaagaaaccaattgtattgttggtgttccggaagcatatggtggtggtggtgatagcagcattccgaccgcaatgggtgttctgtatggtattaaagcaaccaataaaatgctgtttggtaaagatgatctgggtggtgttacctatgcaattcagggtctgggtaaagttggttataaagttgcagaaggtctgctggaagaaggtgcacatctgtttgttaccgatattaatgaacagaccctggaagcaattcaggaaaaagcaaaaaccaccagcggtagcgttaccgttgttgcaagcgatgaaatttatagccaggaagcagatgtttttgttccgtgtgcatttggtggtgttgttaatgatgaaaccatgaaacagtttaaagttaaagcaattgcaggtagcgcaaataatcagctgctgaccgaagatcatggtcgtcatctggcagataaaggtattctgtatgcaccggattatattgttaatagcggtggtctgattcaggttgcagatgaactgtatgaagttaataaagaacgtgttctggcaaaaaccaaacatatttatgatgcaattctggaagtttatcagcaggcagaactggatcagattaccaccatggaagcagcaaatcgtatgtgtgaacagcgtatggcagcacgtggtcgtcgtaatagcttttttaccagcagcgttaaaccgaaatgggatattcgtaattaa

SEQ ID NO: 10 - DNA, 58 bp, synthetic construct
  source 1..58: mol_type = other DNA
gaattcattaaagaggagaaaggtaccatgacagacaagagccaacaattcgccagcg

SEQ ID NO: 11 - DNA, 60 bp, synthetic construct
  source 1..60: mol_type = other DNA
aggtcgacatagttaatttctcctactagttcagccaccaatgatcgtgcggatatccgc

SEQ ID NO: 12 - DNA, 45 bp, synthetic construct
  source 1..45: mol_type = other DNA
ttaaagaggagaaaggtaccatgattgatttacgcagtgataccg

SEQ ID NO: 13 - DNA, 55 bp, synthetic construct
  source 1..55: mol_type = other DNA
cagccatagttaatttctcctactagttcagccaccaatgatcgtgcggatatcc

SEQ ID NO: 14 - DNA, 53 bp, synthetic construct
  source 1..53: mol_type = other DNA
gcgcgttaaactagtaggagaaattaactatggctgactcgcaacccctgtcc

SEQ ID NO: 15 - DNA, 55 bp, synthetic construct
  source 1..55: mol_type = other DNA
ctttcgttttatttgatgcctctagactaacccgccaaaaagaacctgaacgccg

SEQ ID NO: 16 - DNA, 59 bp, synthetic construct
  source 1..59: mol_type = other DNA
catccgcatttaagctagcaggagaaattaactatgagcctggtggaaaaaaccagcat

SEQ ID NO: 17 - DNA, 52 bp, synthetic construct
  source 1..52: mol_type = other DNA
atttgatgcctctagagctagcttaattacgaatatcccatttcggtttaac

SEQ ID NO: 18 - DNA, 53 bp, synthetic construct
  source 1..53: mol_type = other DNA
gaattcattaaagaggagaaaggtaccatgcgcgcactggttagcaaagcacg

SEQ ID NO: 19 - DNA, 54 bp, synthetic construct
  source 1..54: mol_type = other DNA
gcccatagttaatttctcctgctagctcactggccaggaccacgaccacgaatc

SEQ ID NO: 20 - DNA, 55 bp, synthetic construct
  source 1..55: mol_type = other DNA
ccagtgagctagcaggagaaattaactatgggcaaactggacaaagcgagcaaac

SEQ ID NO: 21 - DNA, 56 bp, synthetic construct
  source 1..56: mol_type = other DNA
gacatagttaatttctcctaagctttcaccagccaattttaccggtgtctttcgga

SEQ ID NO: 22 - DNA, 59 bp, synthetic construct
  source 1..59: mol_type = other DNA
ggtggctgaactagtaggagaaattaactatgtcgacctcaccccaccgccccgctcat

SEQ ID NO: 23 - DNA, 64 bp, synthetic construct
  source 1..64: mol_type = other DNA
atagttttgctcatagttaatttctcctgctagctcacggccacgacatgcgatgcagccgagc

SEQ ID NO: 24 - DNA, 55 bp, synthetic construct
  source 1..55: mol_type = other DNA
tggctggtaaaagcttaggagaaattaactatgtcgacctcaccccaccgccccg

SEQ ID NO: 25 - DNA, 51 bp, synthetic construct
  source 1..51: mol_type = other DNA
cgttttatttgatgcctctagatcacggccacgacatgcgatgcagccgag

SEQ ID NO: 26 - DNA, 65 bp, synthetic construct
  source 1..65: mol_type = other DNA
catgtcgtggccgtgagctagcaggagaaattaactatgagcaaaactatcgcgacggaaaatgc

SEQ ID NO: 27 - DNA, 56 bp, synthetic construct
  source 1..56: mol_type = other DNA
gcctttcgttttatttgatgcctctagattagcgacgaacagcgatcgcttcgatc

SEQ ID NO: 28 - DNA, 51 bp, synthetic construct
  source 1..51: mol_type = other DNA
tcattaaagaggagaaaggtaccatggcggtatttgccagtgactcttttg

SEQ ID NO: 29 - DNA, 49 bp, synthetic construct
  source 1..49: mol_type = other DNA
catagttaatttctcctgctagcttaaatgcggatgatggtcgtctttg

(19) United States
(12) Patent Application Publication
(10) Pub. No.: US 2025/0305012 A1
(43) Pub. Date: Oct. 2, 2025

(54) METHOD FOR PREPARING KETO ACIDS, AND USE OF SAME IN PREPARATION OF AMINO ACIDS OR AMINO ACID DERIVATIVES

(71) Applicant: MINT BIOTECHNOLOGIES CO., LTD., Hangzhou, Zhejiang (CN)

(72) Inventors: Mengzhen NIE, Hangzhou, Zhejiang (CN); Kechun ZHANG, Hangzhou, Zhejiang (CN)

(21) Appl. No.: 18/855,803

(22) PCT Filed: Apr. 11, 2023
(86) PCT No.: PCT/CN2023/087476

§ 371 Date: Oct. 10, 2024

(30) Foreign Application Priority Data
Apr. 11, 2022 (CN) 202210371436.6

(51) Int. Cl. C12P 13/00; C12N 9/10; C12N 9/88; C12P 7/42; C12P 7/50; C12P 13/22

(52) U.S. Cl. CPC C12P 13/005; C12N 9/1022; C12N 9/88; C12P 7/42; C12P 7/50; C12P 13/222

(57) ABSTRACT

In a method for preparing a keto acid, an enzymatic reaction is carried out by using glycine and an alcoholic organic substance as substrates; the alcoholic organic substance is converted into an aldehyde organic substance, glycine and the aldehyde organic substance are converted into a β-hydroxy-α-amino acid, and then the β-hydroxy-α-amino acid is converted into a keto acid. The preparation method for a keto acid can also be used in the preparation of amino acids. The number of enzymes used is much less than that of enzymes used in a natural synthesis route, so that the production cost is low. An artificial metabolism platform for keto acids is established and can produce multiple important keto acids, such as phenylpyruvic acid, 4-methyl-2-oxopentanoic acid, pyruvic acid and 2-oxo-butyric acid.

### CROSS-REFERENCE TO RELATED APPLICATIONS

The present disclosure claims priority to Chinese Patent Application No. 202210371436.6 filed to China National Intellectual Property Administration on Apr. 11, 2022 and entitled “METHOD FOR PREPARING KETO ACIDS, AND USE OF SAME IN PREPARATION OF AMINO ACIDS OR AMINO ACID DERIVATIVES”, which is incorporated herein by reference in its entirety.

### TECHNICAL FIELD

The present disclosure relates to the technical field of bioengineering, and particularly relates to a method for preparing a keto acid and use of the method for manufacturing an amino acid.

### BACKGROUND

Amino acids are the basic unit of biological functional macromolecular proteins, and play an important role in life activities of humans and animals as well as in nutrition and health. An amino acid is an organic compound comprising a basic amino group (—NH₂) and an acidic carboxyl group (—COOH), and the amino group and the carboxyl group are attached to the same carbon atom, with a chemical general formula of RCHNH₂COOH. According to the degree of need for the human body, amino acids can be divided into essential amino acids and non-essential amino acids. In addition to biosynthesized natural amino acids, amino acids also include artificially synthesized unnatural amino acids. Amino acid products have a wide range of applications in medical/health care, food (flavorings), feed additives, cosmetics, chemical synthesis, etc., and have become a major global industry.

Currently, the main production methods for amino acids in the world include fermentation method, chemical synthesis method, hydrolysis method, and chemical synthesis-enzymatic method. The fermentation method is a production method that utilizes the inherent ability of microorganisms to synthesize various amino acids, and selects and breeds various defective and resistant mutant strains through strain mutagenesis and other treatments, in order to relieve the feedback and repression in the metabolism pathway, and achieve the overproduction of a certain amino acid. The inevitable disadvantages of the fermentation method for producing amino acids are the difficulty of controlling the production and accumulation of various non-target amino acids, long production period, high difficulty of purification, and high cost. The chemical synthesis method is a method for producing amino acids by means of complicated reactions between various organic substances. Although various natural and unnatural amino acids can be produced by the chemical synthesis method, most of the resulting amino acids are optical racemates (comprising two kinds of optical isomers, D and L), which greatly restricts the industrial value. Meanwhile, the chemical synthesis method also has the problems of complicated reactions, numerous steps, and environmental pollution. The hydrolysis method is a process to obtain various amino acids by using protein-rich materials such as hair as starting materials, subjecting the same to hydrolysis with acid, alkali, or proteolytic enzyme, and then performing separation and purification. The hydrolysis method, with richer starting materials, is relatively easy to put into production, but has low yield, high cost, and relatively serious pollution to the environment. Most of the amino acids can be synthesized using keto acids via a one-step reaction with transaminases, so the production and preparation of keto acids are of great importance. Currently, the problems in the preparation of keto acids are use of toxic catalysts and the phenomenon of low yield and transformation rate. U.S. Pat. No. 8,153,839B2 discloses a method for synthesizing a keto acid or an amino acid by hydration of an acetylene compound, which takes advantage of the hydration of a metal salt and a transition metal coordination complex to allow the synthesis of a keto acid (including a keto acid and a keto acid derivative) from acetylene-carboxylic acid under mild conditions without using an extremely harmful mercury catalyst, and which can easily synthesize an amino acid (including an amino acid and an amino acid derivative) from the synthesized keto acid (including a keto acid and a keto acid derivative) by subsequent reductive amination in the same container. However, the operation thereof is complicated, the production process is difficult to control, and the yield and transformation rate are low, making it difficult to achieve industrial mass production. Therefore, the present disclosure aims to seek a breakthrough in technological innovation for the preparation of keto acids, improve the yield of a plurality of natural amino acids or unnatural amino acids and derivatives in all aspects, and enhance the competitiveness of the product in the market.

### SUMMARY

The first technical problem to be solved by the present disclosure is to provide a method for preparing a keto acid with high substrate utilization rate, low pollution, high yield, and low production cost. The second technical problem to be solved by the present disclosure is to provide a method for preparing a glycol organic substance with high substrate utilization rate, low pollution, high yield, and low production cost.

The third technical problem to be solved by the present disclosure is to be able to produce a plurality of important keto acids: phenylpyruvic acid, 4-methyl-2-oxopentanoic acid, pyruvic acid, 2-oxobutyric acid, etc., and the pathway can be applied to the production of other keto acids and amino acids.

The technical problem solved by the present disclosure is realized by adopting the following technical solutions:

In a first aspect, provided is a method for preparing a keto acid, wherein an enzymatic reaction is performed using glycine and an alcoholic organic substance as substrates, wherein during the enzymatic reaction process, the alcoholic organic substance is converted into an aldehyde organic substance, the glycine and the aldehyde organic substance are converted into a β-hydroxy-α-amino acid, and then the β-hydroxy-α-amino acid is converted into the keto acid.

Preferably, the keto acid prepared comprises, but is not limited to, a keto acid having the following general formula:

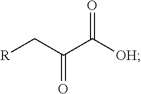

wherein the structural formula of R may be

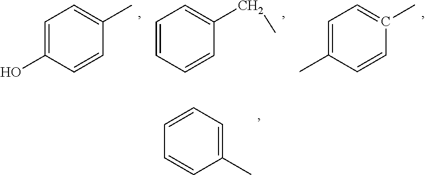

(CH₃)₂CH₂—, (CH₃)C—, CH₃—,

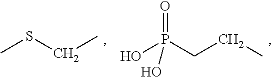

etc.

Further, an enzymatic reaction is performed using glycine and an alcoholic organic substance as substrates and using an enzyme produced by overexpression of a first recombinant microorganism comprising L-aldolase and first dehydratase genes and a second recombinant microorganism comprising a dehydrogenase as a catalyst, wherein the alcoholic organic substance is converted into an aldehyde organic substance in the presence of the dehydrogenase, the glycine and the aldehyde organic substance are converted into a β-hydroxy-α-amino acid under the independent catalysis of the L-aldolase, and the β-hydroxy-α-amino acid generates the keto acid under the catalysis of the first dehydratase;

or an enzymatic reaction is performed using an enzyme produced by overexpression of a third recombinant microorganism comprising a D-aldolase gene, a racemase gene, and a second dehydratase gene and a second recombinant microorganism comprising the dehydrogenase as a catalyst, wherein the alcoholic organic substance is converted into an aldehyde organic substance in the presence of the dehydrogenase, the glycine and the aldehyde organic substance are converted into a β-hydroxy-α-amino acid under the co-catalysis of the D-aldolase and the racemase, and the β-hydroxy-α-amino acid generates the keto acid under the catalysis of the second dehydratase.

Further, the L-aldolase gene is selected from one or more of ltaE, ItaE_Pp, psald, dhaa, CC_3093, fbaA, itaA, glyA, or URA1, and is preferably ltaE_Pp or ItaE, wherein more preferably, the nucleotide sequence of the ltaE_Pp or ItaE gene is set forth in SEQ ID NO. 1 or SEQ ID NO. 2; the first dehydratase gene is selected from one or more of ilvA, tdcB, TDH, CHA1, TD2, A8H32_14290, Saut_1089, and C0627_08730, and is preferably ilvA or A8H32_14290, wherein more preferably, the nucleotide sequence of the ilvA or A8H32_14290 gene is set forth in SEQ ID NO. 3 or SEQ ID NO. 4; the dehydrogenase gene comprises one or more of adhE, adh, ADH7, xylB, adhA, xylW, ped, leuB, BADH, aldh, ACIAD1578, and qbdA, and is preferably xylB, wherein more preferably, the nucleotide sequence of the xylB gene is set forth in SEQ ID NO. 6.

Further, the D-aldolase gene is selected from one or more of A0A1C9ZZ39_CHLRE, tasS, dna, cghG, folB, guaB, dus, dhaa, bhcC, NCTC12151_01614, A4G23_03658, OJAG_33340, and GGC03_18995, and is preferably A0A1C9ZZ39_CHLRE, wherein more preferably, the nucleotide sequence of the A0A1C9ZZ39_CHLRE gene is set forth in SEQ ID NO. 7; the racemase gene is selected from one or more of ILE2E_LENBU, agiA, puuE, PS659_05479, HRbin10_02390, CVS47_02795, HRbin08_01795, and MJ8_44540, and is preferably ILE2E_LENBU, wherein more preferably, the nucleotide sequence of the ILE2E_LENBU gene is set forth in SEQ ID NO. 8; the second dehydratase gene is selected from one or more of ilvA, tdcB, TDH, CHA1, TD2, A8H32_14290, Saut_1089, and C0627_08730, wherein more preferably, the nucleotide sequence of the ilvA gene is set forth in SEQ ID NO. 3.

Further, the first recombinant microorganism further comprises an enamine/imine deaminase gene, and is preferably ridA, wherein more preferably, the nucleotide sequence of the ridA gene is set forth in SEQ ID NO: 5.

Further, the alcoholic organic substance is selected from one or more of benzyl alcohol, 4-imidazolemethanol, 2-(methylthio) ethanol, indole-3-methanol, 2-hydroxyethyl-methyl phosphinic acid, p-hydroxybenzyl alcohol, 3,4-dihydroxybenzyl alcohol, p-methylbenzyl alcohol, phenethyl alcohol, tert-amyl alcohol, isobutanol, and ethanol.

In a second aspect, provided is a method for preparing a keto acid, wherein an enzymatic reaction is performed using glycine and an aldehyde organic substance as substrates, wherein during the enzymatic reaction process, the glycine and the aldehyde organic substance are converted into a β-hydroxy-α-amino acid, and then the β-hydroxy-α-amino acid is converted into the keto acid. Preferably, the keto acid prepared comprises, but is not limited to, a keto acid having the following general formula:

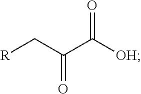

wherein the structural formula of R may be

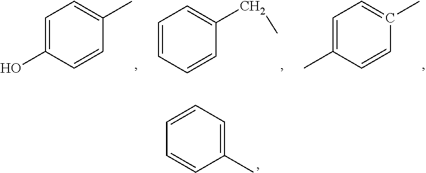

(CH₃)₂CH₂—, (CH₃)C—, CH₃—,

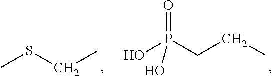

etc.

Further, an enzymatic reaction is performed using glycine and an aldehyde organic substance as substrates and using an enzyme produced by overexpression of a first recombinant microorganism comprising L-aldolase and first dehydratase genes as a catalyst, wherein the glycine and the aldehyde organic substance are converted into a β-hydroxy-α-amino acid under the independent catalysis of the L-aldolase, and the β-hydroxy-α-amino acid generates the keto acid under the catalysis of the first dehydratase;

or an enzymatic reaction is performed using glycine and an aldehyde organic substance as substrates and using an enzyme produced by overexpression of the third recombinant microorganism comprising a D-aldolase gene, a racemase gene, and a second dehydratase gene as a catalyst, wherein the glycine and the aldehyde organic substance are converted into a β-hydroxy-α-amino acid under the co-catalysis of the D-aldolase and the racemase, and the β-hydroxy-α-amino acid generates the keto acid under the catalysis of the second dehydratase.

Further, the L-aldolase gene is selected from one or more of ltaE, ItaE_Pp, psald, dhaa, CC_3093, fbaA, itaA, glyA, or URA1, and is preferably ItaE_Pp or ItaE, wherein more preferably, the nucleotide sequence of the ltaE_Pp or ltaE gene is set forth in SEQ ID NO. 1 or SEQ ID NO. 2; the first dehydratase gene is selected from one or more of ilvA, tdcB, TDH, CHA1, TD2, A8H32_14290, Saut_1089, and C0627_08730, and is preferably ilvA or A8H32_14290, wherein more preferably, the nucleotide sequence of the ilvA or A8H32_14290 gene is set forth in SEQ ID NO. 3 or SEQ ID NO. 4.

Further, the D-aldolase gene is selected from one or more of A0A1C9ZZ39_CHLRE, tasS, dna, cghG, folB, guaB, dus, dhaa, bhcC, NCTC12151_01614, A4G23_03658, OJAG_33340, and GGC03_18995, and is preferably A0A1C9ZZ39_CHLRE, wherein more preferably, the nucleotide sequence of the A0A1C9ZZ39_CHLRE gene is set forth in SEQ ID NO. 7; the racemase gene is selected from one or more of ILE2E_LENBU, agiA, puuE, PS659_05479, HRbin10_02390, CVS47_02795, HRbin08_01795, and MJ8_44540, and is preferably ILE2E_LENBU, wherein more preferably, the nucleotide sequence of the ILE2E_LENBU gene is set forth in SEQ ID NO. 8; the second dehydratase gene is selected from one or more of ilvA, tdcB, TDH, CHA1, TD2, A8H32_14290, Saut_1089, and C0627_08730, wherein more preferably, the nucleotide sequence of the ilvA gene is set forth in SEQ ID NO. 3.

Further, the construction of the first recombinant microorganism, the second recombinant microorganism, or the third recombinant microorganism by a genetic engineering method is included, and the genetic engineering method includes plasmid expression or genomic integration. Further, in a case that the construction is performed by means of plasmid expression, the plasmid vector used is selected from one or two of pZAlac and pZElac.

Further, the constructed recombinant microorganism is cultured and then subjected to an enzymatic reaction, wherein the culturing method for the recombinant microorganism is: inoculating the recombinant microorganism into a 2-xyT culture medium comprising ampicillin, kanamycin, and chloramphenicol, culturing at 20-60° C. for 3-6 h, adding IPTG to a final concentration of 0.3 mM, culturing for another 15-30 h and then centrifuging, and decanting the culture supernatant.

Further, during the enzymatic reaction process, the reaction temperature is 20-90° C., and the pH of the reaction buffer is 6.5-8.5.

Further, the recombinant microorganism comprises one or more of recombinant Escherichia coli, Bacillus, Corynebacterium, Saccharomyces, or Streptomyces.

Further, the recombinant microorganism is selected from one or more of recombinant Escherichia coli, Bacillus subtilis, Pseudomonas sp., Bacillus megaterium, Bacillus amyloliquefaciens, Corynebacterium glutamicum, Saccharomyces cerevisiae, Candida utilis, or Pichia pastoris.

In a third aspect, provided is a recombinant microorganism for preparing a keto acid, wherein the recombinant microorganism is a first recombinant microorganism comprising L-aldolase and first dehydratase genes, and a second recombinant microorganism comprising a dehydrogenase; or the recombinant microorganism is a third recombinant microorganism comprising a D-aldolase gene, a racemase gene, and a second dehydratase gene, and a second recombinant microorganism comprising a dehydrogenase.

Preferably, the L-aldolase gene is selected from one or more of ltaE, ItaE_Pp, psald, dhaa, CC_3093, fbaA, itaA, glyA, or URA1, and is preferably ltaE_Pp or ItaE, wherein more preferably, the nucleotide sequence of the ltaE_Pp or ItaE gene is set forth in SEQ ID NO. 1 or SEQ ID NO. 2; the first dehydratase gene is selected from one or more of ilvA, tdcB, TDH, CHA1, TD2, A8H32_14290, Saut_1089, and C0627_08730, and is preferably ilvA or A8H32_14290, wherein more preferably, the nucleotide sequence of the ilvA or A8H32_14290 gene is set forth in SEQ ID NO. 3 or SEQ ID NO. 4; the dehydrogenase gene is selected from one or more of adhE, adh, ADH7, xylB, adhA, xylW, ped, leuB, BADH, aldh, ACIAD1578, and qbdA, and is preferably xylB, wherein more preferably, the nucleotide sequence of the xylB gene is set forth in SEQ ID NO. 6.

Alternatively, the D-aldolase gene is selected from one or more of A0A1C9ZZ39_CHLRE, tasS, dna, cghG, folB, guaB, dus, dhaa, bhcC, NCTC12151_01614, A4G23_03658, OJAG_33340, and GGC03_18995, and is preferably A0A1C9ZZ39_CHLRE, wherein more preferably, the nucleotide sequence of the A0A1C9ZZ39_CHLRE gene is set forth in SEQ ID NO. 7; the racemase gene is selected from one or more of ILE2E_LENBU, agiA, puuE, PS659_05479, HRbin10_02390, CVS47_02795, HRbin08_01795, and MJ8_44540, and is preferably ILE2E_LENBU, wherein more preferably, the nucleotide sequence of the ILE2E_LENBU gene is set forth in SEQ ID NO. 8; the second dehydratase gene is selected from one or more of ilvA, tdcB, TDH, CHA1, TD2, A8H32_14290, Saut_1089, and C0627_08730, wherein more preferably, the nucleotide sequence of the ilvA gene is set forth in SEQ ID NO. 3.

Further preferably, the first recombinant microorganism further comprises an enamine/imine deaminase gene, and is preferably ridA, wherein more preferably, the nucleotide sequence of the ridA gene is set forth in SEQ ID NO: 5.

In a fourth aspect, provided is a recombinant microorganism for preparing a keto acid, wherein the recombinant microorganism is a first recombinant microorganism comprising L-aldolase and first dehydratase genes; or the recombinant microorganism is a third recombinant microorganism comprising a D-aldolase gene, a racemase gene, and a second dehydratase gene;
preferably, the L-aldolase gene is selected from one or more of ltaE, ItaE_Pp, psald, dhaa, CC_3093, fbaA, itaA, glyA, or URA1, and is preferably ltaE_Pp or ItaE, wherein more preferably, the nucleotide sequence of the ltaE_Pp or ItaE gene is set forth in SEQ ID NO. 1 or SEQ ID NO. 2; the first dehydratase gene is selected from one or more of ilvA, tdcB, TDH, CHA1, TD2, A8H32_14290, Saut_1089, and C0627_08730, and is preferably ilvA or A8H32_14290, wherein more preferably, the nucleotide sequence of the ilvA or A8H32_14290 gene is set forth in SEQ ID NO. 3 or SEQ ID NO. 4;

alternatively, the D-aldolase gene is selected from one or more of A0A1C9ZZ39_CHLRE, tasS, dna, cghG, folB, guaB, dus, dhaa, bhcC, NCTC12151_01614, A4G23_03658, OJAG_33340, and GGC03_18995, and is preferably A0A1C9ZZ39_CHLRE, wherein more preferably, the nucleotide sequence of the A0A1C9ZZ39_CHLRE gene is set forth in SEQ ID NO. 7; the racemase gene is selected from one or more of ILE2E_LENBU, agiA, puuE, PS659_05479, HRbin10_02390, CVS47_02795, HRbin08_01795, and MJ8_44540, and is preferably ILE2E_LENBU, wherein more preferably, the nucleotide sequence of the ILE2E_LENBU gene is set forth in SEQ ID NO. 8; the second dehydratase gene is selected from one or more of ilvA, tdcB, TDH, CHA1, TD2, A8H32_14290, Saut_1089, and C0627_08730, wherein more preferably, the nucleotide sequence of the ilvA gene is set forth in SEQ ID NO. 3.

Further, the keto acid comprises, but is not limited to, a keto acid having the following general formula:

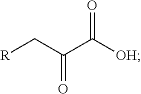

wherein the structural formula of R may be

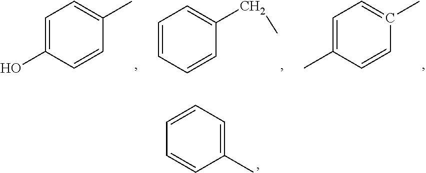

(CH₃)₂CH₂—, (CH₃)C—, CH₃—,

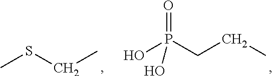

etc.

Further, the recombinant microorganism is selected from one or more of recombinant Escherichia coli, Bacillus subtilis, Pseudomonas sp., Bacillus megaterium, Bacillus amyloliquefaciens, Corynebacterium glutamicum, Saccharomyces cerevisiae, Candida utilis, or Pichia pastoris.

In a fifth aspect, provided is a recombinant DNA or biomaterial for use in preparing a keto acid,
wherein the recombinant DNA or biomaterial
comprises L-aldolase and first dehydratase genes, and comprises a dehydrogenase gene; or the recombinant DNA or biomaterial comprises a D-aldolase gene, a racemase gene, and a second dehydratase gene, and comprises a dehydrogenase;
preferably, the L-aldolase gene is selected from one or more of ltaE, ItaE_Pp, psald, dhaa, CC_3093, fbaA, itaA, glyA, or URA1, and is preferably ltaE_Pp or ItaE, wherein more preferably, the nucleotide sequence of the ltaE_Pp or ItaE gene is set forth in SEQ ID NO. 1 or SEQ ID NO. 2; the first dehydratase gene is selected from one or more of ilvA, tdcB, TDH, CHA1, TD2, A8H32_14290, Saut_1089, and C0627_08730, and is preferably ilvA or A8H32_14290, wherein more preferably, the nucleotide sequence of the ilvA or A8H32_14290 gene is set forth in SEQ ID NO. 3 or SEQ ID NO. 4; the dehydrogenase gene is selected from one or more of adhE, adh, ADH7, xylB, adhA, xylW, ped, leuB, BADH, aldh, ACIAD1578, and qbdA, and is preferably xylB, wherein more preferably, the nucleotide sequence of the xylB gene is set forth in SEQ ID NO. 6;
alternatively, the D-aldolase gene is selected from one or more of A0A1C9ZZ39_CHLRE, tasS, dna, cghG, folB, guaB, dus, dhaa, bhcC, NCTC12151_01614, A4G23_03658, OJAG_33340, and GGC03_18995, and is preferably A0A1C9ZZ39_CHLRE, wherein more preferably, the nucleotide sequence of the A0A1C9ZZ39_CHLRE gene is set forth in SEQ ID NO. 7; the racemase gene is selected from one or more of ILE2E_LENBU, agiA, puuE, PS659_05479, HRbin10_02390, CVS47_02795, HRbin08_01795, and MJ8_44540, and is preferably ILE2E_LENBU, wherein more preferably, the nucleotide sequence of the ILE2E_LENBU gene is set forth in SEQ ID NO. 8; the second dehydratase gene is selected from one or more of ilvA, tdcB, TDH, CHA1, TD2, A8H32_14290, Saut_1089, and C0627_08730, wherein more preferably, the nucleotide sequence of the ilvA gene is set forth in SEQ ID NO. 3;
further preferably, the recombinant DNA or biomaterial further comprises an enamine/imine deaminase gene, and is preferably ridA, wherein more preferably, the nucleotide sequence of the ridA gene is set forth in SEQ ID NO: 5;
preferably, the biomaterial is an expression cassette, a transposon, a plasmid vector, a phage vector, or a virus vector.

In a sixth aspect, provided is a recombinant DNA or biomaterial for use in preparing a keto acid, wherein the recombinant DNA or biomaterial comprises L-aldolase and first dehydratase genes; or the recombinant DNA or biomaterial comprises a D-aldolase gene, a racemase gene, and a second dehydratase gene;
preferably, the L-aldolase gene is selected from one or more of ltaE, ItaE_Pp, psald, dhaa, CC_3093, fbaA, itaA, glyA, or URA1, and is preferably ltaE_Pp or ItaE, wherein more preferably, the nucleotide sequence of the ltaE_Pp or ItaE gene is set forth in SEQ ID NO. 1 or SEQ ID NO. 2; the first dehydratase gene is selected from one or more of ilvA, tdcB, TDH, CHA1, TD2, A8H32_14290, Saut_1089, and C0627_08730, and is preferably ilvA or A8H32_14290, wherein more preferably, the nucleotide sequence of the ilvA or A8H32_14290 gene is set forth in SEQ ID NO. 3 or SEQ ID NO. 4;
alternatively, the D-aldolase gene is selected from one or more of A0A1C9ZZ39_CHLRE, tasS, dna, cghG, folB, guaB, dus, dhaa, bhcC, NCTC12151_01614, A4G23_03658, OJAG_33340, and GGC03_18995, and is preferably A0A1C9ZZ39_CHLRE, wherein more preferably, the nucleotide sequence of the A0A1C9ZZ39_CHLRE gene is set forth in SEQ ID NO. 7; the racemase gene is selected from one or more of ILE2E_LENBU, agiA, puuE, PS659_05479, HRbin10_02390, CVS47_02795, HRbin08_01795, and MJ8_44540, and is preferably ILE2E_LENBU, wherein more preferably, the nucleotide sequence of the ILE2E_LENBU gene is set forth in SEQ ID NO. 8; the second dehydratase gene is selected from one or more of ilvA, tdcB, TDH, CHA1, TD2, A8H32_14290, Saut_1089, and C0627_08730, wherein more preferably, the nucleotide sequence of the ilvA gene is set forth in SEQ ID NO. 3.

Preferably, the biomaterial is an expression cassette, a transposon, a plasmid vector, a phage vector, or a virus vector.

Further, the keto acid comprises, but is not limited to, a keto acid having the following general formula:

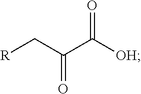

wherein the structural formula of R may be

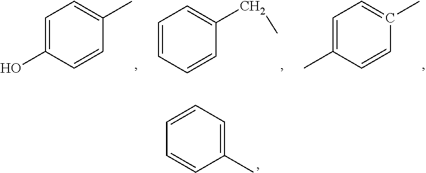

(CH₃)₂CH₂—, (CH₃)C—, CH₃—,

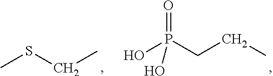

etc.

In a seventh aspect, provided is use of the recombinant microorganism for preparing a keto acid according to the third or fourth aspect, and the recombinant DNA or biomaterial for preparing a keto acid according to the fifth or sixth aspect for manufacturing a keto acid.

In an eighth aspect, provided is use of the recombinant microorganism for preparing a keto acid according to the third or fourth aspect, and the recombinant DNA or biomaterial for preparing a keto acid according to the fifth or sixth aspect for manufacturing an amino acid or an amino acid derivative.

In a ninth aspect, provided is a method for preparing a glycol organic substance, wherein the glycol organic substance is generated using the β-hydroxy-α-amino acid obtained by the method according to the first aspect in the presence of a deaminase, a decarboxylase, and a reductase.

### Beneficial Effects:

According to the method for preparing a keto acid described herein, by providing a novel multi-enzyme catalytic system, an enzymatic reaction is performed using glycine and different kinds of aldehydes in the presence of the multi-enzyme catalytic system. The number of the enzymes used herein is greatly less than that of a natural synthetic route, and the production cost is low. In addition, an artificial metabolic platform for keto acids is constructed, such that a plurality of important keto acids can be produced, e.g., phenylpyruvic acid, 4-methyl-2-oxopentanoic acid, pyruvic acid, and 2-oxobutyric acid, with simple operation, high yield, high substrate utilization rate, and low pollution.

The method for preparing a keto acid described herein is applied to the preparation process of amino acids and amino acid derivatives, achieving the preparation of a plurality of amino acids and amino acid derivatives with high output and high yield, and achieving industrial mass production.

The present disclosure is also applicable to the preparation of glycol organic substances.

### BRIEF DESCRIPTION OF THE DRAWINGS

FIG. 1 shows a graph of the process for producing phenylpyruvic acid from benzaldehyde and glycine in the R2 system.

FIG. 2 shows a graph of the process for producing 4-methyl-2-oxopentanoic acid from isobutyraldehyde and glycine in the R3 and R4 systems.

FIG. 3 shows a graph of the process for producing 2-oxobutyric acid from acetaldehyde and glycine in the R5 system.

### DETAILED DESCRIPTION

In order to make the technical means, creation characteristics, achieved purposes, and effects of the present disclosure easy to understand, the present disclosure is further illustrated below with reference to specific examples.

In the present disclosure, unless otherwise indicated, the scientific and technical terms used herein have the same meaning as commonly understood by those skilled in the art. In addition, the terms and laboratory procedures related to nucleic acid chemistry, molecular biology, cell and tissue culture, microbiology, and immunology used herein are the terms and conventional procedures widely used in the corresponding fields. Also, in order to better understand the present disclosure, the definitions and interpretations of the related terms are provided below.

It should be appreciated that the terms used herein are for the purpose of illustrating particular embodiments only, and are not intended to be limiting.

The articles “a”, “an” and “the” are used herein to refer to one or more than one of the grammatical object of the article.

The use of alternatives (e.g., “or”) should be understood to refer to one, two, or any combination of the alternatives. The term “and/or” should be interpreted as referring to one or both of the alternatives. As used herein, the term “gene synthesis” refers to a generation process using recombinant DNA techniques or a production process using DNA or amino acid sequence synthesis techniques available and well known in the art.

The term “encode” or “code” refers to the inherent property of specific sequences of nucleotides in a polynucleotide, such as a gene, a cDNA, or an mRNA, to serve as a template in synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of an mRNA corresponding to the gene produces the protein in a cell or other biological system. Both the coding strand, which comprises a nucleotide sequence equivalent to the mRNA sequence and is usually provided in a sequence listing, and the non-coding strand, which is used as a template for transcription of a gene or cDNA, may be referred to as encoding the protein or other product of that gene or cDNA.

As used herein, the term “endogenous” refers to any substance derived from or produced within an organism, cell, tissue or system.

As used herein, the term “exogenous” refers to any substance introduced into or produced outside of an organism, cell, tissue or system.

As used herein, the term “expression” is defined as the transcription and/or translation of a particular nucleotide sequence driven by its promoter.

Unless otherwise specified, the “polynucleotide sequence encoding an amino acid sequence” includes all nucleotide sequences that are degenerate versions of each other and encode the same amino acid sequence. The phrase “nucleotide sequence encoding a protein or an RNA” may also include introns to the extent that the nucleotide sequence encoding the protein may contain an intron(s) in some versions.

As used herein, the term “vector” refers to a composition of matter that comprises an isolated nucleic acid and can be used to deliver the isolated nucleic acid to the interior of a cell. The transferred nucleic acid is typically ligated to, e.g., inserted into, a vector nucleic acid molecule. A vector may comprise sequences that direct autonomous replication in the cell, or may comprise sequences sufficient to allow integration into the host cell DNA. Many vectors are known in the art, including but not limited to plasmids, phagemids, artificial chromosomes, bacteriophages and animal viruses. Therefore, the term “vector” encompasses an autonomously replicating plasmid or virus.

In some embodiments, the L-aldolase-encoding gene comprises the ItaE gene, the fbaA gene of Bacillus subtilis, the psald gene of Pseudomonas, etc., and the dehydratase gene comprises the ilvA gene of Escherichia coli, the thadh gene of Pseudomonas aeruginosa, the A8H32_14290_14160 gene of Burkholderia cepacia, the thrc gene of Bacillus subtilis, etc. A8H32_14290 denotes the dehydratase A8H32_14290_14160 from Burkholderia cepacia.

The gene may be wild-type or mutant gene. For example, in some embodiments, the A8H32_14290 gene may be wild-type A8H32_14290 or mutant A8H32_14290m, wherein the mutant A8H32_14290m refers to a A8H32_14290 mutant gene using PLP as a coenzyme, and the mutant A8H32_14290 is different from wild-type A8H32_14290 using PLP as a coenzyme. The mutant A8H32_14290 carries mutations S246A and E291A.

In some embodiments, the L-aldolase (ItaE), dehydratase (ilvA, tdcB), and enamine/imine deaminase (ridA) are from Escherichia coli genome.

In some embodiments, the L-aldolase (ltaE) may be from Pseudomonas putida, and the L-aldolase from Pseudomonas putida is denoted as ItaE-Pp herein.

In some embodiments, the dehydratase (A8H32_14290) may be from Burkholderia cepacia.

The genes described above are obtained by PCR reaction using DNA primers.

The vector used herein comprises the polynucleotide molecule according to any one of the preceding items, wherein the vector further comprises common elements including, but not limited to, one or more of an origin of replication (ORI), an antibiotic resistance gene, a multiple cloning site (MCS), a promoter, an enhancer, a primer binding site, and a selectable marker.

In some embodiments, the vector is a plasmid vector, wherein the plasmid is one or two of pZAlac and pZElac.

A primer is designed based on the genomic sequence of Pseudomonas putida published by NCBI:

ltaE_Pp-F:
(SEQ ID NO: 10)
GAATTCATTAAAGAGGAGAAAGGTACCATGACAGACAAGAGCCAACAAT
TCGCCAGCG;
ltaE_Pp-R:
(SEQ ID NO: 11)
AGGTCGACATAGTTAATTTCTCCTACTAGTTCAGCCACCAATGATCGTG
CGGATATCCGC

A primer was designed based on the genomic sequence of Escherichia coli MG1655 published by NCBI:

ltaE-F:
(SEQ ID NO: 12)
TTAAAGAGGAGAAAGGTACCATGATTGATTTACGCAGTGATACCG;
ltaE-R:
(SEQ ID NO: 13)
CAGCCATAGTTAATTTCTCCTACTAGTTCAGCCACCAATGATCGTGCGG
ATATCC;
ilvA-F:
(SEQ ID NO: 14)
GCGCGTTAAACTAGTAGGAGAAATTAACTATGGCTGACTCGCAACCCCT
GTCC);
ilvA-R:
(SEQ ID NO: 15)
CTTTCGTTTTATTTGATGCCTCTAGACTAACCCGCCAAAAAGAACCTGA
ACGCCG.

A primer is designed based on the genomic sequence of Bacillus (Bacillus badius) published by NCBI:

pdh-F:
(SEQ ID NO: 16)
CATCCGCATTTAAGCTAGCAGGAGAAATTAACTATGAGCCTGGTGGAAA
AAACCAGCAT;
pdh-R:
(SEQ ID NO: 17)
ATTTGATGCCTCTAGAGCTAGCTTAATTACGAATATCCCATTTCGGTTT
AAC.

A primer is designed based on the genomic sequence of Chlamydomonas reinhardtii published by NCBI:

A0A1C9ZZ39 CHLRE-F:
(SEQ ID NO: 18)
GAATTCATTAAAGAGGAGAAAGGTACCATGCGCGCACTGGTTAGCAAAG
CACG;
A0A1C9ZZ39 CHLRE-R:
(SEQ ID NO: 19)
GCCCATAGTTAATTTCTCCTgctagcTCACTGGCCAGGACCACGACCAC
GAATC.

A primer is designed based on the genomic sequence of Lactobacillus buchneri published by NCBI:

ILE2E_LENBU-F:
(SEQ ID NO: 20)
CCAGTGAGCTAGCAGGAGAAATTAACTATGGGCAAACTGGACAAAGCGA
GCAAAC;
ILE2E_LENBU-R:
(SEQ ID NO: 21)
GACATAGTTAATTTCTCCTAAGCTTTCACCAGCCAATTTTACCGGTGTC
TTTCGGA.

Two groups of primers were designed based on the genomic sequence of Burkholderia cepacia (Burkholderia thailandensis E264) published by NCBI:

A8H32_14290-F:
(SEQ ID NO: 22)
GGTGGCTGAACTAGTAGGAGAAATTAACTATGTCGACCTCACCCCACCG
CCCCGCTCAT;
A8H32_14290-R:
(SEQ ID NO: 23)
ATAGTTTTGCTCATAGTTAATTTCTCCTGCTAGCTCACGGCCACGACAT
GCGATGCAGCCGAGC;
A8H32_14290'-F:
(SEQ ID NO: 24)
TGGCTGGTAAAAGCTTAGGAGAAATTAACTATGTCGACCTCACCCCACC
GCCCCG;
A8H32_14290'-R:
(SEQ ID NO: 25)
CGTTTTATTTGATGCCTCTAGATCACGGCCACGACATGCGATGCAGCCG
AG.

A primer was designed based on the genomic sequence of Escherichia coli MG1655 published by NCBI:

ridA-F:
(SEQ ID NO: 26)
CATGTCGTGGCCGTGAGCTAGCAGGAGAAATTAACTATGAGCAAAACTA
TCGCGACGGAAAATGC;
ridA-R:
(SEQ ID NO: 27)
GCCTTTCGTTTTATTTGATGCCTCTAGATTAGCGACGAACAGCGATCGC
TTCGATC.

A primer is designed based on the genomic sequence of Pseudomonas putida published by NCBI:

xylB-F:
(SEQ ID NO: 28)
TCATTAAAGAGGAGAAAGGTACCATGGCGGTATTTGCCAGTGACTCTTT
TG;
xylB-R:
(SEQ ID NO: 29)
CATAGTTAATTTCTCCTGCTAGCTTAAATGCGGATGATGGTCGTCTTT
G.

The corresponding gene fragments are obtained by PCR, digested by restriction endonuclease, and then ligated to plasmid pZElac through a ligase to give recombinant expression plasmid vectors pZE-ltaE_Pp-A8H32_14290, pZE-ItaE_Pp-A8H32_14290-ridA, pZE-ItaE-ilvA, pZE-ItaE-ilvA-ridA, pZA-xylB-pdh, and pZE-A0A1C9ZZ39_CHLRE-ILE2E_LENBU-A8H32_14290′.

Among them:

The nucleotide sequence of the ltaE_Pp gene is set forth in SEQ ID NO: 1.

The nucleotide sequence of the ItaE gene is set forth in SEQ ID NO: 2.

The nucleotide sequence of the ilvA gene is set forth in SEQ ID NO: 3.

The nucleotide sequence of the A8H32_14290 gene is set forth in SEQ ID NO: 4.

The nucleotide sequence of the ridA gene is set forth in SEQ ID NO: 5.

The nucleotide sequence of the xylB gene is set forth in SEQ ID NO: 6.

The nucleotide sequence of the A0A1C9ZZ39_CHLRE gene is set forth in SEQ ID NO: 7.

The nucleotide sequence of the ILE2E_LENBU gene is set forth in SEQ ID NO: 8.

The nucleotide sequence of the pdh gene used in Example 4 is set forth in SEQ ID NO: 9.

### Example 1

In this example, glycine and benzaldehyde were used as starting materials, and the product was phenylpyruvic acid.

The recombinant expression plasmid vectors pZE-ItaE_Pp-A8H32_14290 and pZE-ItaE_Pp-A8H32_14290-ridA were transformed into an expression strain Escherichia coli BL21 separately to give recombinant Escherichia coli marked as R1 and R2. The recombinant Escherichia coli was inoculated into a 2-xyT culture medium containing ampicillin and chloramphenicol, and the mixture was cultured in a 50 mL Erlenmeyer flask (liquid volume 10 mL) at 30° C. with a rotation speed of 240 rpm for 3-6 h. IPTG was added to a final concentration of 0.3 mM, and the mixture was cultured for another 20 h to enable protein expression. The mixture was centrifuged at 4° C. with a rotation speed of 8000 rpm for 5 min, and the supernatant culture medium was decanted to give a bacterial solution. The bacterial solution was subjected to an ice bath for further use.

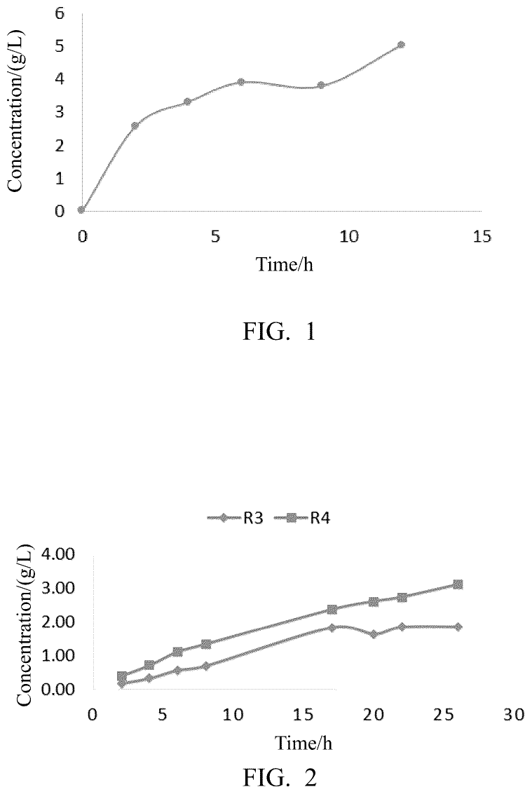

The bacterial solution described above was added to a 2 mL system containing 50 mM Tris-HCl buffer at pH 7.5. Glycine was added at a ratio of 20 g/L (the ratio of glycine to the system), and benzaldehyde (4.6 g/L) was added. The mixture was allowed to react under shaking in a shaker at 30° C. and 240 rpm for 24 h to give a transformation solution containing phenylpyruvic acid. Samples were taken every 2 h of catalysis to detect the generation of phenylpyruvic acid. The obtained transformation solution was diluted 20-fold with deionized water, and centrifuged at 12500 rpm/min for 10-15 min. The supernatant was pipetted and diluted, and high performance liquid chromatography analysis was performed. The concentrations of the substrates and product in the catalytic system were determined. According to the determination, 3.6 g/L phenylpyruvic acid was produced in the R1 system after 24 h of catalytic reaction, and 5 g/L phenylpyruvic acid was produced in the R2 system after 12 h of catalytic reaction. In the R2 system, the addition of the ridA gene could accelerate the conversion from the intermediate enamine to the final product, thus making the reaction faster and increasing the concentration and yield of the product (as shown in FIG. 1).

### Example 2

In this example, glycine and isobutyraldehyde were used as starting materials, and the product was 4-methyl-2-oxopentanoic acid.

The recombinant expression plasmid vectors pZE-ItaE-ilvA and pZE-ItaE-ilvA-ridA were transformed into an expression strain Escherichia coli BL21 separately to give recombinant Escherichia coli marked as R3 and R4. The recombinant Escherichia coli was inoculated into a 2-xyT culture medium containing ampicillin and chloramphenicol, and the mixture was cultured in a 50 mL Erlenmeyer flask (liquid volume 10 mL) at 30° C. with a rotation speed of 240 rpm for 3-6 h. IPTG was added to a final concentration of 0.3 mM, and the mixture was cultured for another 20 h. Then, the mixture was centrifuged at 4° C. with a rotation speed of 8000 rpm for 5 min, and the supernatant culture medium was decanted to give a bacterial solution. The bacterial solution was subjected to an ice bath for further use.

The bacterial solution described above was added to a 2 mL system containing 50 mM Tris-HCl buffer (pH 7.5), 20 g/L glycine, and 2.6 g/L isobutyraldehyde. The mixture was allowed to react under shaking in a shaker at 30° C. and 240 rpm for 24 h to give a transformation solution containing oxopentanoic acid. Samples were taken every 2 h of catalysis to detect the generation of oxopentanoic acid. The obtained transformation solution was diluted 20-fold with deionized water, and centrifuged at 12500 rpm/min for 10-15 min. The supernatant was pipetted and diluted, and high performance liquid chromatography analysis was performed. The concentration of the product in the catalytic system was determined. According to the determination, after 26 h of catalytic reaction, 1.9 g/L 4-methyl-2-oxopentanoic acid was produced in the R3 system, and 3.1 g/L 4-methyl-2-oxopentanoic acid was produced in the R4 system. The addition of the ridA gene could accelerate the conversion from the intermediate enamine to the final product, thus making the reaction faster and increasing the concentration and yield of the product (as shown in FIG. 2).

### Example 3

In this example, glycine and acetaldehyde were used as starting materials, and the product was 2-oxobutyric acid.

The recombinant expression plasmid vector pZE-ItaE-ilvA was transformed into an expression strain Escherichia coli BL21 to give recombinant Escherichia coli marked as R5. The recombinant Escherichia coli was inoculated into a 2-xyT culture medium containing ampicillin and chloramphenicol, and the mixture was cultured in a 50 mL Erlenmeyer flask (liquid volume 10 mL) at 30° C. with a rotation speed of 240 rpm for 3-6 h. IPTG was added to a final concentration of 0.3 mM, and the mixture was cultured for another 20 h. The mixture was centrifuged at 4° C. with a rotation speed of 8000 rpm for 5 min, and the supernatant culture medium was decanted to give a bacterial solution. The bacterial solution was subjected to an ice bath for further use.

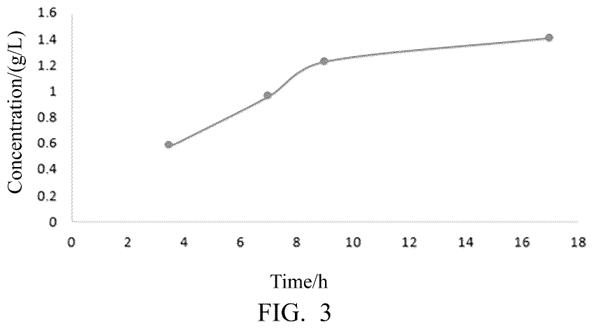

The bacterial solution described above was added to a 2 mL system containing 50 mM Tris-HCl buffer at pH 8.0, and 20 g/L glycine and 15 mL acetaldehyde were added. The mixture was fermented in a shaker at 30° C. and 240 rpm, and allowed to react under shaking for 3 h to give a transformation solution containing 2-oxobutyric acid. The obtained transformation solution was diluted 10-fold with deionized water, and centrifuged at 12500 rpm/min for 10-15 min. The supernatant was pipetted and diluted, and high performance liquid chromatography analysis was performed. The concentration of the product was determined. According to the determination, after 18 h of catalytic reaction, a total of 1.4 g/L 2-oxobutyric acid was produced (as shown in FIG. 3).

### Example 4

In this example, glycine and benzyl alcohol were used as starting materials, and the products were phenylpyruvic acid and phenylalanine.

The recombinant expression plasmid vectors pZE-ItaE_Pp-A8H32_14290 and pZA-xylB-pdh were electroporated into an expression strain Escherichia coli BL21 simultaneously to give recombinant Escherichia coli marked as R6. (The pdh gene encodes an enzyme which reduces phenylpyruvic acid to phenylalanine, and in this example, the substrate keto acid was further converted to an amino acid). The strain was inoculated into a 2-xyT culture medium containing ampicillin, kanamycin, and chloramphenicol, and the mixture was cultured in a 50 mL Erlenmeyer flask (liquid volume 10 mL) at 30° C. with a rotation speed of 240 rpm for 3-6 h. IPTG was added to a final concentration of 0.3 mM, and the mixture was cultured for another 20 h. The mixture was centrifuged at 4° C. with a rotation speed of 8000 rpm for 5 min, and the supernatant culture medium was decanted to give a bacterial solution. The bacterial solution was subjected to an ice bath for further use.

The bacterial solution described above was added to a 2 mL system containing 50 mM Tris-HCl buffer at pH 7.5. Glycine was added at a ratio of 20 g/L, and benzyl alcohol was added at a ratio of 4.6 g/L. The mixture was fermented in a shaker at 30° C. and 240 rpm, and allowed to react under shaking for 24 h to give a transformation solution containing phenylalanine. After 24 h of catalytic reaction, the obtained transformation solution was diluted 20-fold with deionized water, and centrifuged at 12500 rpm/min for 10-15 min. The supernatant was pipetted and diluted, and high performance liquid chromatography analysis was performed. The concentrations of the substrates and products in the catalytic system were determined. According to the determination, 2.3 g/L phenylpyruvic acid and 1.2 g/L phenylalanine were produced in the R6 system.

### Example 5

In this example, glycine and benzaldehyde were used as starting materials, and the product was phenylpyruvic acid (D-aldolase).

The recombinant expression plasmid vector pZE-A0A1C9ZZ39_CHLRE-ILE2E LENBU-A8H32_14290′ was transformed into an expression strain Escherichia coli BL21 to give recombinant Escherichia coli marked as R7. The recombinant Escherichia coli was inoculated into a 2-xyT culture medium containing ampicillin and chloramphenicol, and the mixture was cultured in a 50 mL Erlenmeyer flask (liquid volume 10 mL) at 30° C. with a rotation speed of 240 rpm for 3-6 h. IPTG was added to a final concentration of 0.3 mM, and the mixture was cultured for another 20 h to enable protein expression. The mixture was centrifuged at 4° C. with a rotation speed of 8000 rpm for 5 min, and the supernatant culture medium was decanted to give a bacterial solution. The bacterial solution was subjected to an ice bath for further use.

The bacterial solution described above was added to a 2 mL system containing 50 mM Tris-HCl buffer at pH 7.5. Glycine was added at a ratio of 20 g/L (the ratio of glycine to the system), and 4.6 g/L benzaldehyde was added. The mixture was allowed to react under shaking in a shaker at 30° C. and 240 rpm for 24 h to give a transformation solution containing phenylpyruvic acid. Samples were taken every 2 h of catalysis to detect the generation of phenylpyruvic acid. The obtained transformation solution was diluted 20-fold with deionized water, and centrifuged at 12500 rpm/min for 10-15 min. The supernatant was pipetted and diluted, and high performance liquid chromatography analysis was performed. The concentrations of the substrates and product in the catalytic system were determined. According to the determination, after 24 h of catalytic reaction, 2.6 g/L phenylpyruvic acid was produced in the R7 system.

The foregoing shows and describes the general principles, principal features, and advantages of the present disclosure. It should be understood by those skilled in the art that the present disclosure is not limited to the examples described above, and the examples described above and the description in the specification are merely illustration of the principles of the present disclosure. Various changes and modifications may be made without departing from the spirit and scope of the present disclosure, and such changes and modifications are within the protection scope of the present disclosure as claimed. The present disclosure claims a scope of protection as defined by the appended claims and the equivalents thereof.

1. A method for preparing a keto acid, wherein an enzymatic reaction is performed using glycine and an alcoholic organic substance as substrates, wherein during the enzymatic reaction process, the alcoholic organic substance is converted into an aldehyde organic substance, the glycine and the aldehyde organic substance are converted into a β-hydroxy-α-amino acid, and then the β-hydroxy-α-amino acid is converted into the keto acid.

2. The method as claimed in claim 1, wherein the keto acid prepared comprises, but is not limited to, a keto acid having the following general formula: 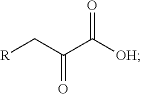
wherein the structural formula of R may be 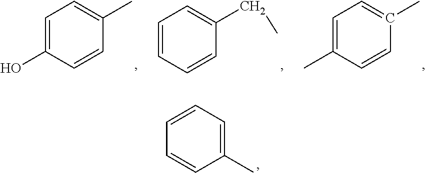
(CH₃)₂CH₂—, (CH₃)C—, CH₃—, 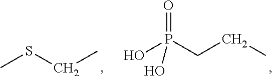
etc.

3. The method for preparing a keto acid as claimed in claim 1, wherein an enzymatic reaction is performed using glycine and an alcoholic organic substance as substrates and using an enzyme produced by overexpression of a first recombinant microorganism comprising L-aldolase and first dehydratase genes and a second recombinant microorganism comprising a dehydrogenase as a catalyst, wherein the alcoholic organic substance is converted into an aldehyde organic substance in the presence of the dehydrogenase, the glycine and the aldehyde organic substance are converted into a β-hydroxy-α-amino acid under the independent catalysis of the L-aldolase, and the β-hydroxy-α-amino acid generates the keto acid under the catalysis of the first dehydratase;
or, an enzymatic reaction is performed using an enzyme produced by overexpression of a third recombinant microorganism comprising a D-aldolase gene, a racemase gene, and a second dehydratase gene and a second recombinant microorganism comprising the dehydrogenase as a catalyst, wherein the alcoholic organic substance is converted into an aldehyde organic substance in the presence of the dehydrogenase, the glycine and the aldehyde organic substance are converted into a β-hydroxy-α-amino acid under the co-catalysis of the D-aldolase and the racemase, and the β-hydroxy-α-amino acid generates the keto acid under the catalysis of the second dehydratase.

4. The method for preparing a keto acid as claimed in claim 3, wherein the L-aldolase gene is selected from one or more of ltaE, ItaE_Pp, psald, dhaa, CC_3093, fbaA, itaA, glyA, or URA1, and is preferably ltaE_Pp or ItaE, wherein more preferably, the nucleotide sequence of the ltaE_Pp or ltaE gene is set forth in SEQ ID NO. 1 or SEQ ID NO. 2; the first dehydratase gene is selected from one or more of ilvA, tdcB, TDH, CHA1, TD2, A8H32_14290, Saut_1089, and C0627_08730, and is preferably ilvA or A8H32_14290, wherein more preferably, the nucleotide sequence of the ilvA or A8H32_14290 gene is set forth in SEQ ID NO. 3 or SEQ ID NO. 4; the dehydrogenase gene comprises one or more of adhE, adh, ADH7, xylB, adhA, xylW, ped, leuB, BADH, aldh, ACIAD1578, and qbdA, and is preferably xylB, wherein more preferably, the nucleotide sequence of the xylB gene is set forth in SEQ ID NO. 6.

5. The method for preparing a keto acid as claimed in claim 3, wherein the D-aldolase gene is selected from one or more of A0A1C9ZZ39_CHLRE, tasS, dna, cghG, folB, guaB, dus, dhaa, bhcC, NCTC12151_01614, A4G23_03658, OJAG_33340, and GGC03_18995, and is preferably A0A1C9ZZ39_CHLRE, wherein more preferably, the nucleotide sequence of the A0A1C9ZZ39_CHLRE gene is set forth in SEQ ID NO. 7; the racemase gene is selected from one or more of ILE2E_LENBU, agiA, puuE, PS659_05479, HRbin10_02390, CVS47_02795, HRbin08_01795, and MJ8_44540, and is preferably ILE2E_LENBU, wherein more preferably, the nucleotide sequence of the ILE2E_LENBU gene is set forth in SEQ ID NO. 8; the second dehydratase gene is selected from one or more of ilvA, tdcB, TDH, CHA1, TD2, A8H32_14290, Saut_1089, and C0627_08730, wherein more preferably, the nucleotide sequence of the ilvA gene is set forth in SEQ ID NO. 3.

6. The method for preparing a keto acid as claimed in claim 5, wherein the first recombinant microorganism further comprises an enamine/imine deaminase gene, and is preferably ridA, wherein more preferably, the nucleotide sequence of the ridA gene is set forth in SEQ ID NO: 5.

7. The method for preparing a keto acid as claimed in claim 6, wherein the alcoholic organic substance is selected from one or more of benzyl alcohol, 4-imidazolemethanol, 2-(methylthio) ethanol, indole-3-methanol, 2-hydroxyethyl-methyl phosphinic acid, p-hydroxybenzyl alcohol, 3,4-dihydroxybenzyl alcohol, p-methylbenzyl alcohol, phenethyl alcohol, tert-amyl alcohol, isobutanol, and ethanol.

8. A method for preparing a keto acid, wherein an enzymatic reaction is performed using glycine and an aldehyde organic substance as substrates, wherein during the enzymatic reaction process, the glycine and the aldehyde organic substance are converted into a β-hydroxy-α-amino acid, and then the β-hydroxy-α-amino acid is converted into the keto acid.

9. The method as claimed in claim 8, wherein the keto acid prepared comprises, but is not limited to, a keto acid having the following general formula: 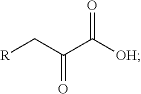
wherein the structural formula of R may be 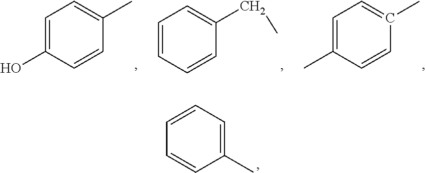
(CH₃)₂CH₂—, (CH₃)C—, CH₃—, 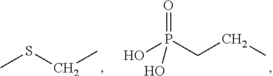
etc.

10. The method as claimed in claim 8, wherein an enzymatic reaction is performed using glycine and an aldehyde organic substance as substrates and using an enzyme produced by overexpression of a first recombinant microorganism comprising L-aldolase and first dehydratase genes as a catalyst, wherein the glycine and the aldehyde organic substance are converted into a β-hydroxy-α-amino acid under the independent catalysis of the L-aldolase, and the β-hydroxy-α-amino acid generates the keto acid under the catalysis of the first dehydratase;
or an enzymatic reaction is performed using glycine and an aldehyde organic substance as substrates and using an enzyme produced by overexpression of the third recombinant microorganism comprising a D-aldolase gene, a racemase gene, and a second dehydratase gene as a catalyst, wherein the glycine and the aldehyde organic substance are converted into a β-hydroxy-α-amino acid under the co-catalysis of the D-aldolase and the racemase, and the β-hydroxy-α-amino acid generates the keto acid under the catalysis of the second dehydratase.

11. The method as claimed in claim 10, wherein the L-aldolase gene is selected from one or more of ltaE, ItaE_Pp, psald, dhaa, CC_3093, fbaA, itaA, glyA, or URA1, and is preferably ItaE_Pp or ltaE, wherein more preferably, the nucleotide sequence of the ltaE_Pp or ItaE gene is set forth in SEQ ID NO. 1 or SEQ ID NO. 2;
the first dehydratase gene is selected from one or more of ilvA, tdcB, TDH, CHA1, TD2, A8H32_14290, Saut_1089, and C0627_08730, and is preferably ilvA or A8H32_14290, wherein more preferably, the nucleotide sequence of the ilvA or A8H32_14290 gene is set forth in SEQ ID NO. 3 or SEQ ID NO. 4.

12. The method as claimed in claim 10, wherein the D-aldolase gene is selected from one or more of A0A1C9ZZ39_CHLRE, tasS, dna, cghG, folB, guaB, dus, dhaa, bhcC, NCTC12151_01614, A4G23_03658, OJAG_33340, and GGC03_18995, and is preferably A0A1C9ZZ39_CHLRE, wherein more preferably, the nucleotide sequence of the A0A1C9ZZ39_CHLRE gene is set forth in SEQ ID NO. 7; the racemase gene is selected from one or more of ILE2E_LENBU, agiA, puuE, PS659_05479, HRbin10_02390, CVS47_02795, HRbin08_01795, and MJ8_44540, and is preferably ILE2E_LENBU, wherein more preferably, the nucleotide sequence of the ILE2E_LENBU gene is set forth in SEQ ID NO. 8; the second dehydratase gene is selected from one or more of ilvA, tdcB, TDH, CHA1, TD2, A8H32_14290, Saut_1089, and C0627_08730, wherein more preferably, the nucleotide sequence of the ilvA gene is set forth in SEQ ID NO. 3.

13. The method as claimed in claim 10, wherein the construction of the first recombinant microorganism, the second recombinant microorganism, or the third recombinant microorganism by a genetic engineering method is included, and the genetic engineering method includes plasmid expression or genomic integration.

14. The method as claimed in claim 13, wherein in a case that the construction is performed by means of plasmid expression, the plasmid vector used is selected from one or two of pZAlac and pZElac.

15. The method as claimed in claim 14, wherein the constructed recombinant microorganism is cultured and then subjected to an enzymatic reaction, wherein the culturing method for the recombinant microorganism is: inoculating the recombinant microorganism into a 2-xyT culture medium comprising ampicillin, kanamycin, and chloramphenicol, culturing at 20-60° C. for 3-6 h, adding IPTG to a final concentration of 0.3 mM, culturing for another 15-30 h and then centrifuging, and decanting the supernatant culture medium.

16. The method as claimed in claim 15, wherein during the enzymatic reaction process, the reaction temperature is 20-90° C., and the pH of the reaction buffer is 6.5-8.5.

17. The preparation method for a keto acid as claimed in claim 16, wherein the recombinant microorganism comprises one or more of recombinant Escherichia coli, Bacillus, Corynebacterium, Saccharomyces, or Streptomyces.

18. (canceled)

19. A recombinant microorganism for preparing a keto acid, wherein the recombinant microorganism is a first recombinant microorganism comprising L-aldolase and first dehydratase genes, and a second recombinant microorganism comprising a dehydrogenase; or, the recombinant microorganism is a third recombinant microorganism comprising a D-aldolase gene, a racemase gene, and a second dehydratase gene, and a second recombinant microorganism comprising a dehydrogenase;
preferably, the L-aldolase gene is selected from one or more of ltaE, ItaE_Pp, psald, dhaa, CC_3093, fbaA, itaA, glyA, or URA1, and is preferably ItaE_Pp or ItaE, wherein more preferably, the nucleotide sequence of the ltaE_Pp or ItaE gene is set forth in SEQ ID NO. 1 or SEQ ID NO. 2; the first dehydratase gene is selected from one or more of ilvA, tdcB, TDH, CHA1, TD2, A8H32_14290, Saut_1089, and C0627_08730, and is preferably ilvA or A8H32_14290, wherein more preferably, the nucleotide sequence of the ilvA or A8H32_14290 gene is set forth in SEQ ID NO. 3 or SEQ ID NO. 4; the dehydrogenase gene is selected from one or more of adhE, adh, ADH7, xylB, adhA, xylW, ped, leuB, BADH, aldh, ACIAD1578, and qbdA, and is preferably xylB, wherein more preferably, the nucleotide sequence of the xylB gene is set forth in SEQ ID NO. 6;
alternatively, the D-aldolase gene is selected from one or more of A0A1C9ZZ39_CHLRE, tasS, dna, cghG, folB, guaB, dus, dhaa, bhcC, NCTC12151_01614, A4G23_03658, OJAG_33340, and GGC03_18995, and is preferably A0A1C9ZZ39_CHLRE, wherein more preferably, the nucleotide sequence of the A0A1C9ZZ39_CHLRE gene is set forth in SEQ ID NO. 7; the racemase gene is selected from one or more of ILE2E_LENBU, agiA, puuE, PS659_05479, HRbin10_02390, CVS47_02795, HRbin08_01795, and MJ8_44540, and is preferably ILE2E_LENBU, wherein more preferably, the nucleotide sequence of the ILE2E_LENBU gene is set forth in SEQ ID NO. 8; the second dehydratase gene is selected from one or more of ilvA, tdcB, TDH, CHA1, TD2, A8H32_14290, Saut_1089, and C0627_08730, wherein more preferably, the nucleotide sequence of the ilvA gene is set forth in SEQ ID NO. 3;
further preferably, the first recombinant microorganism further comprises an enamine/imine deaminase gene, and is preferably ridA, wherein more preferably, the nucleotide sequence of the ridA gene is set forth in SEQ ID NO: 5.

20-28. (canceled)
